(19) United States
(12) Patent Application Publication
(10) Pub. No.: US 2020/0087275 A1
(43) Pub. Date: Mar. 19, 2020

(54) GIBBERELLIC ACID COMPOSITIONS

(71) Applicant: GROSPURT ENTERPRISES INC., Vancouver (CA)

(72) Inventor: Kenneth CURRY, Vancouver (CA)

(21) Appl. No.: 16/570,677

(22) Filed: Sep. 13, 2019

Related U.S. Application Data
(60) Provisional application No. 62/733,452, filed on Sep. 19, 2018.

(51) Int. Cl. C07D 307/93

(52) U.S. Cl. CPC C07D 307/93

(57) ABSTRACT

The present disclosure provides an anhydrous solution that includes between 5% and 15% w/w of gibberellic acid (GA3) in a solvent, where the solvent consists of: (a) isopropanol; (b) up to 10% w/w of methanol, ethanol, or a combination thereof; (c) up to 10% w/w of dimethyl sulfoxide (DMSO); and (d) up to 50% w/w of n-propanol, tetrahydrofurfuryl alcohol (THFA), or a combination thereof. The disclosure also provides methods of making such solutions.

### CROSS REFERENCE TO RELATED APPLICATIONS

This application claims the benefit of priority of U.S. Provisional Patent Application No. 62/733,452 filed Sep. 19, 2018, which is hereby incorporated by reference.

### FIELD

The present disclosure relates to solutions that include gibberellic acid.

### BACKGROUND

The following paragraphs are not an admission that anything discussed in them is prior art or part of the knowledge of persons skilled in the art.

Gibberellin A3 (“GA3” or “gibberellic acid”) is a plant growth hormone that has a chemical structure according to Formula I:

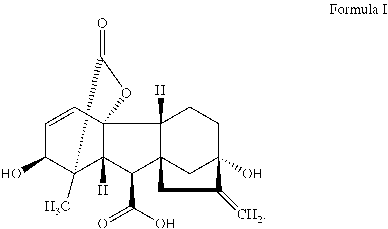

Gibberellic acid may be used to trigger germination in seeds that would otherwise remain dormant. It may be used to induce the production of larger bundles and bigger grapes, such as Thompson seedless grapes. It is may be used as a growth replicator in the cherry industry. It may be applied directly to the blossoms of Clementine Mandarin oranges to induce production of fruit.

### INTRODUCTION

The following introduction is intended to introduce the reader to this specification but not to define any invention. One or more inventions may reside in a combination or sub-combination of the apparatus elements or method steps described below or in other parts of this document. The inventors do not waive or disclaim their rights to any invention or inventions disclosed in this specification merely by not describing such other invention or inventions in the claims.

Although gibberellin A3 is soluble in water and is available in solid form, it is also sold commercially as an anhydrous solution since the chemical may undergo hydrolysis in aqueous or aqueous-alcoholic solutions. GA3 is sold by Grospurt Canada as a solution of 4 g GA3 per 100 mL (78.5 g) of isopropyl alcohol (also referred to as “IPA” or “isopropanol”). This corresponds to a solution of 4.8 wt % GA in IPA.

The authors of the present disclosure have surprisingly discovered that concentrations of 5 wt % to 15 wt % of GA3 in an anhydrous solvent may be achieved if the GA3 is dissolved in the anhydrous solvent at an elevated temperature, and the resulting solution is allowed to cool to room temperature. Exemplary solutions are stable to addition of a seed crystal of GA3, cooling to −20° C., or both. In the context of the present disclosure, a solution that is “stable” to a given condition should be understood to refer to a solution that does not crystallize under the noted condition.

In one aspect, the present disclosure provides an anhydrous solution that includes between 5 wt % and 15 wt % of gibberellic acid (GA3) in a solvent, where the solvent consists of: (a) isopropanol; (b) up to 10% w/w of methanol, ethanol, or a combination thereof; (c) up to 10% w/w of dimethyl sulfoxide (DMSO); and (d) up to 50% w/w of n-propanol, tetrahydrofurfuryl alcohol (THFA), or a combination thereof.

In specific embodiments, the solvent consists of at least 95% w/w isopropanol. For example, the solvent may be isopropanol.

The solution may comprise between 7 wt % and 11 wt %, such as about 10 wt %, of the GA3.

In another aspect, the present disclosure provides a method of making an anhydrous solution that includes gibberellic acid (GA3) in an anhydrous solvent. The method includes: dissolving a mixture of the GA3 in the anhydrous solvent at an elevated temperature sufficient to dissolve the GA3; and allowing the resulting solution to cool to room temperature. The mixture includes between 5 wt % and 15 wt % of the gibberellic acid (GA3). The solvent consists of: (a) isopropanol; (b) up to 10% w/w of methanol, ethanol, or a combination thereof; (c) up to 10% w/w of dimethyl sulfoxide (DMSO); and (d) up to 50% w/w of n-propanol, tetrahydrofurfuryl alcohol (THFA), or a combination thereof.

The temperature may be greater than 50° C. and less than the boiling point of the solvent.

In embodiments where the solvent is isopropanol, (i) the mixture may include about 5 wt % of GA3, and the method may include dissolving the GA3 in the solvent at a temperature of about 52° C.; (ii) the mixture may include from about 8 wt % to about 10 wt % of GA3, and the method may include dissolving the GA3 in the solvent at a temperature of about 62° C.; (iii) the mixture may include about 12 wt % of GA3, and the method may include dissolving the GA3 in the solvent at a temperature of about 64° C.; or (iv) the mixture may include about 15 wt % of GA3, and the method may include dissolving the GA3 in the solvent at a temperature of about 75° C.

In embodiments where the solvent is a mixture of 95% isopropanol and 5% DMSO w/w, the mixture may include about 15 wt % of the GA3, and the method may include dissolving the GA3 in the solvent at a temperature of about 65° C.

### DETAILED DESCRIPTION

Generally, the present disclosure provides an anhydrous solution that includes between 5 wt % and 15 wt % of gibberellic acid (GA3) in a solvent, where the solvent consists of: (a) isopropanol; (b) up to 10% w/w of methanol; ethanol, or a combination thereof; (c) up to 10% w/w of dimethyl sulfoxide (DMSO); and (d) up to 50% w/w of n-propanol, tetrahydrofurfuryl alcohol (THEA), or a combination thereof.

In some examples, the solvent consist of isopropanol and DMSO; or isopropanol and THEA. The solvent may consists of at least 95% w/w isopropanol. In particular examples, the solvent is isopropanol.

The solution may include between 7 wt % and 11 wt %, such as about 10 wt %, of GA3.

The solution may consist essentially of the GA3 and the solvent. For example, the solution may consist of the GA3, the solvent; and any chemical components present in a technical grade of the GA3.

The present disclosure also provides a method of making an anhydrous solution that includes gibberellic acid (GA3) in an anhydrous solvent. The method includes: dissolving a mixture of the GA3 in the anhydrous solvent at an elevated temperature sufficient to dissolve the GA3; and allowing the resulting solution to cool to room temperature. The mixture includes between 5 wt % and 15 wt % of the gibberellic acid (GA3). The solvent consists of: (a) isopropanol; (b) up to 10% w/w of methanol, ethanol, or a combination thereof; (c) up to 10% w/w of dimethyl sulfoxide (DMSO); and (d) up to 50% w/w of n-propanol, tetrahydrofurfuryl alcohol (THEA), or a combination thereof.

The method may include mixing the GA3 in the solvent and heating the mixture to the elevated temperature, or may include adding the GA to a solvent at the elevated temperature.

The temperature may be greater than 50° C. and less than the boiling point of the solvent.

In some examples; the solvent consists of isopropanol and DMSO; or isopropanol and THFA. The solvent may consist of at least 95% w/w isopropanol. In particular examples, the solvent is isopropanol.

The solution may include between 7 wt % and 11 wt %, such as about 10 wt %, of GA3.

The solution may consist essentially of the GA3 and the solvent. For example, the solution may consist of the GA3, the solvent, and any chemical components present in a technical grade of the GA3.

In embodiments where the solvent is isopropanol, (i) the mixture may include about 5 wt % of GA3, and the method may include dissolving the GA3 in the solvent at a temperature of about 52 CC; (ii) the mixture may include from about 8 wt % to about 10 wt % of GA3, and the method may include dissolving the GA3 in the solvent at a temperature of about 62° C.; (iii) the mixture may include about 12 wt % of GA3, and the method may include dissolving the GA3 in the solvent at a temperature of about 64° C.; or (iv) the mixture may include about 15 wt % of GA3, and the method may include dissolving the GA3 in the solvent at a temperature of about 75° C.

In embodiments where the solvent is a mixture of 95% isopropanol and 5% DMSO w/w, the mixture may include about 15 wt % of the GA3, and the method may include dissolving the GA3 in the solvent at a temperature of about 65° C.

In the context of the present disclosure, it should be understood that the expressions “# wt %” and “# % w/w” are both calculated based on the total weight. For example, a solution that is # wt % of a solute is calculated by dividing the weight of the solute by the weight of the solution (including the solute; the solvent(s), and any other components). Similarly, a solution that is made up of “# % w/w” of one solvent refers to the weight of the solvent divided by the total weight of all the solvents. For example, a solvent with 10 g of methanol and 90 g of isopropanol could be referred to as 10% w/w of methanol.

In the context of the present disclosure, an “elevated temperature” should be understood to refer to a temperature higher than room temperature and below the boiling point of the solvent or mixture of solvents.

In the context of the present disclosure, the term “about” should be understood to mean “nearly” or “approximately”. In some circumstances; the variation may be based on the accuracy of a typical instrument used to measure the value. For example, when referring to a temperature of a solvent, “about 50° C.” would be understood to refer to temperatures from 49° C. to 51° C. since typical glass thermometers used to measure the temperature of liquids have an accuracy of about +/−1° C., In other circumstances, the variation may be based on the precision of the reported value. For example, a mixture of “about 10 wt %” of a solid dissolved in a solvent would be understood to refer to mixtures from 9.5 wt % to 10.49 wt % since “about 9 wt %” would refer to mixtures from 8.5 wt % to 9.49 wt %.

Experimental Results

Different amounts of technical grade GA3 were dissolved in various solvents at various temperatures by mixing the GA3 in the solvent and heating the mixture to the point at which complete solubility was obtained and noting that temperature. The technical grade GA3 was 92% pure. The solvents were ACS quality, IPA 99.5% purity and obtained from VWR International.

A summary of the results are shown in Table 1, which lists the grams of pure GA3 present in the solution, as well as the grams of the technical grade GA3. Mixtures of solvents are shown in weight ratios.

TABLE 1

| | GA3 | GA3 | | | | |
|---|---|---|---|---|---|---|
| Exper- | (grams, | (grams, | | Weight | Temp. | |
| iment | pure) | tech) | solvent | % | (° C.) | Notes |
| 1 | 5 | 5.43 | 94.57 g IPA | 5 | 52 | Fully |
| | | | | | | dissolved |
| 2 | 6 | 6.52 | 93.48 g IPA | 6 | 55 | Fully |
| | | | | | | dissolved |
| 3 | 8 | 8.69 | 91.31 g IPA | 8 | 62 | Fully |
| | | | | | | dissolved |
| 4 | 10 | 10.87 | 89.13 g IPA | 10 | 65 | Fully |
| | | | | | | dissolved |
| 5 | 10 | 10.87 | 89.13 g of | 10 | 62 | Fully |
| | | | IPA:DMSO | | | dissolved |
| | | | 95:5 w/w | | | |
| 6 | 12 | 13.04 | 86.96 g IPA | 12 | 64 | Fully |
| | | | | | | dissolved |
| 7 | 12 | 13.04 | 86.96 g of | 12 | 65 | Fully |
| | | | IPA:DMSO | | | dissolved |
| | | | 95:5 w/w | | | |
| 8 | 15 | 16.3 | 83.7 g IPA | 15 | 75 | Fully |
| | | | | | | dissolved |
| 9 | 20 | 21.74 | 78.26 g IPA | 20 | 82 | Not fully |
| | | | | | | dissolved |

Experiments 1 to 5 and 7 to 9 resulted in solutions that were stable at temperature as low as −20° C. The GA3 precipitated out of the solution of Experiment 7 when cooled to −20° C., though the GA3 re-dissolved into the solution on warming to room temperature. The solutions of Experiments 4 to 8 were treated at room temperature with 0.1 g or 10 mg of GA3, or with 5 mg MgSO₄, as seed crystals. The solutions of Experiments 3, 4, and 5 were stable. GA3 in the solution of Experiment 6 crystalized on addition of the GA3 seed crystals, but not on addition of the MgSO₄ seed crystals. The solution of Experiment 7 dissolved the added GA3 seed crystals. Based on this result, the authors believe that solvent mixtures that include DMSO, such as up to 10% DMSO can dissolve more GA3 than the corresponding solvent mixture without the DMSO.

In the preceding description, for purposes of explanation, numerous details are set forth in order to provide a thorough understanding of the examples. However, it will be apparent to one skilled in the art that these specific details are not required. Accordingly, what has been described is merely illustrative of the application of the described examples and numerous modifications and variations are possible in light of the above teachings.

Since the above description provides examples, it will be appreciated that modifications and variations can be effected to the particular examples by those of skill in the art. Accordingly, the scope of the claims should not be limited by the particular examples set forth herein, but should be construed in a manner consistent with the specification as a whole.

What is claimed is:

1. An anhydrous solution comprising:
between 5 wt % and 15 wt % of gibberellic acid (GA3) in a solvent;
wherein the solvent consists of:
isopropanol;
up to 10% w/w of methanol, ethanol, or a combination thereof;
up to 10% w/w of dimethyl sulfoxide (DMSO); and
up to 50% w/w of n-propanol, tetrahydrofurfuryl alcohol (THFA), or a combination thereof.

2. The anhydrous solution according to claim 1, wherein the solvent consists of isopropanol and DMSO; or of isopropanol and THFA.

3. The anhydrous solution according to claim 1, wherein the solvent consists of at least 95% w/w isopropanol.

4. The anhydrous solution according to claim 1, wherein the solvent is isopropanol.

5. The anhydrous solution according to claim 1, wherein the solution comprises between 7 wt % and 11 wt %, such as about 10 wt %, of GA3.

6. The anhydrous solution according to claim 1, wherein the solution consists essentially of the GA3 and the solvent.

7. The anhydrous solution according to claim 1, wherein the solution consists of the GA3, the solvent, and any chemical components present in a technical grade of the GA3.

8. A method of making an anhydrous solution comprising gibberellic acid (GA3) in a solvent, the method comprising:
dissolving the GA3 in the anhydrous solvent at an elevated temperature sufficient to dissolve the GA3; and
allowing the resulting solution to cool to room temperature,
wherein:
the mixture comprises between 5 wt % and 15 wt % of the gibberellic acid (GA3); and
the solvent consists of:
isopropanol;
up to 10% w/w of methanol; ethanol, or a combination thereof;
up to 10% w/w of dimethyl sulfoxide (DMSO); and
up to 50% w/w of n-propanol, tetrahydrofurfuryl alcohol (THFA), or a combination thereof.

9. The method according to claim 8, wherein the GA3 is added to the anhydrous solvent and the mixture is heated to the elevated temperature, or wherein the anhydrous solvent is at the elevated temperature and the GA3 is added to the anhydrous solvent.

10. The method according to claim 8, wherein the elevated temperature is greater than 50° C. and less than the boiling point of the solvent.

11. The method according to claim 8, wherein the solvent consists of isopropanol and DMSO; or of isopropanol and THFA.

12. The method according to claim 8, wherein the solvent consists of at least 95% w/w isopropanol.

13. The method according to claim 8, wherein the solvent is isopropanol.

14. The method according to claim 8, wherein the solution comprises between 7 wt % and 11 wt %, such as about 10 wt %, of GA3.

15. The method according to claim 8, wherein the solution consists essentially of the GA3 and the solvent.

16. The method according to claim 8, wherein the solution consists of the GA3, the solvent, and any chemical components present in a technical grade of the GA3.

17. The method according to claim 8, wherein:
the solvent is isopropanol, the mixture comprises about 5 wt % of GA3, and the method comprises dissolving the GA3 in the solvent at a temperature of about 52° C.;
the solvent is isopropanol, the mixture comprises from about 8 wt % to about 10 wt of GA3, and the method comprises dissolving the GA3 in the solvent at a temperature of about 62° C.;
the solvent is isopropanol, the mixture comprises about 12 wt % of GA3, and the method comprises dissolving the GA3 in the solvent at a temperature of about 64° C.;
the solvent is isopropanol, the mixture comprises about 15 wt % of GA3, and the method comprises dissolving the GA3 in the solvent at a temperature of about 75° C.; or
the solvent is a mixture of 95% isopropanol and 5% DMSO w/w, the mixture comprises about 15% w/w of the GA3, and the method comprises dissolving the GA3 in the solvent at a temperature of about 65° C.
